(19) United States
(12) Patent Application Publication
(10) Pub. No.: US 2020/0121744 A1
(43) Pub. Date: Apr. 23, 2020

(54) COMPOSITIONS AND METHODS FOR TREATING SKIN DISEASES AND MAINTAINING HEALTHY SKIN

(71) Applicant: The Regents of the University of California, Oakland, CA (US)

(72) Inventors: Huiying Li, Los Angeles, CA (US); Emma Barnard, Los Angeles, CA (US); Baochen Shi, Los Angeles, CA (US)

(21) Appl. No.: 16/347,706

(22) PCT Filed: Nov. 7, 2017
(86) PCT No.: PCT/US17/60319

§ 371 Date: May 6, 2019

Related U.S. Application Data
(60) Provisional application No. 62/471,578, filed on Mar. 15, 2017.
(60) Provisional application No. 62/419,182, filed on Nov. 8, 2016.

(51) Int. Cl. A61K 35/76; A61K 35/74; A61P 17/10

(52) U.S. Cl. CPC A61K 35/76; A61P 17/10; A61K 35/74

(57) ABSTRACT

Provided herein are methods and compositions for the treatment or prevention of a skin disease (e.g., acne) in a subject by administering to the subject a composition comprising a strain of P. granulosum, a strain of P. avidum, and/or a strain of P. humerusii. Also provided herein are methods of determining whether a subject has a skin condition, the method comprising measuring the amount of P. granulosum on the skin of the subject.

### RELATED APPLICATIONS

This application is a § 371 national-stage application based on International Application No. PCT/US17/60319, filed Nov. 7, 2017, which claims the benefit of priority to U.S. Provisional Patent Application Ser. No. 62/419,182, filed Nov. 8, 2016, and U.S. Provisional Patent Application Ser. No. 62/471,578, filed on Mar. 15, 2017, each of which are herein incorporated by reference in their entireties.

### GOVERNMENT SUPPORT

This invention was made with Government support under Grant Number GM099530, awarded by the National Institutes of Health. The Government has certain rights in the invention.

### BACKGROUND

The skin is the largest organ in the human body and functions as the first line of defense by providing a protective barrier between the environment and inner body. The skin harbors several hundreds of resident microorganisms, which function in communities and protect the body from invasion of pathogens. Several studies have shown that shifts in the skin microbiota are associated with various skin diseases.

Acne vulgaris (commonly called acne) is the most common skin disease, affecting 80-85% of the population. It is most prevalent in adolescents and rarely occurs in people over the age of 50. Although acne is not life threatening, it can lead to severe pain and scarring on the skin, and has profoundly negative psychosocial effects. Acne is a disease of the pilosebaceous unit (commonly known as the hair follicle). While its etiology is still unclear with multiple factors involved, the Gram-positive lipophilic anaerobe Propionibacterium acnes has been thought to play a role in acne pathogenesis.

### SUMMARY

In some aspects, provided herein are methods and compositions related to treating or preventing a skin disease (e.g., inflammatory skin disease, such as acne, rosacea, or Porphyria Cutanea Tarda (PCT)), preventing and/or slowing skin aging (e.g., preventing the formation of wrinkles), and maintaining healthy skin by administering to the subject a composition disclosed herein. In some aspects, provided herein are methods of treating or preventing a skin condition in a subject and/or reducing the amount of porphyrins on the skin of a subject by administering (e.g., a subject with symptoms of skin aging, or a subject with a skin condition, such as acne, rosacea, or PCT) a composition comprising at least one strain of P. granulosum, at least one strain of P. avidum, and/or at least one strain of P. humerusii to the subject. The strain of P. granulosum may be HL078PG1 and/or HL082PG1. The strain of P. avidum may be HL063PV1, HL083PV1, and HL307PV1. The strain of P. humerusii may be HL044PA1.

In some aspects, the invention relates, at least in part, to determining whether a subject is at risk for a skin disease (e.g., inflammatory skin disease, such as acne, rosacea, or Porphyria Cutanea Tarda (PCT)), comprising obtaining a skin sample from the subject, optionally isolating microbial DNA from the skin sample and identifying the amount of P. granulosum in the microbiome of the skin sample. In some embodiments, if the microbiome is less than 5%, (e.g., less than 4%, less than 3%, less than 2%, less than 1%, or less than 0.5%) P. granulosum, the subject is considered at risk for a skin disease. In some embodiments, the methods further comprise administering to the subject a composition comprising P. granulosum if P. granulosum is less than 5% (e.g., less than 4%, less than 3%, less than 2%, less than 1%, or less than 0.5%) of the microbiome of the skin sample.

In some embodiments, provided herein are methods determining whether a subject has acne by obtaining a skin sample from the subject, sequencing DNA from the skin sample, and analyzing the DNA for the enrichment of one or more metagenomic elements in Table 1 associated with acne or the depletion of one or more metagenomic elements in Table 1 associated with health, wherein the subject is determined as having acne if one or more of the metagenomic elements in Table 1 associated with acne is enriched in the skin sample or one or more of the metagenomic elements in Table 1 associated with health is depleted in the skin sample. In some embodiments, provided herein are methods for determining whether a subject has acne by obtaining a skin sample from the subject, optionally isolating bacterial DNA from the skin sample, using one or more primer sets to amplify the DNA, using one or more probes to detect the amplified DNA; and analyzing the probe signals for the enrichment of one or more metagenomic elements in Table 1 associated with acne or the depletion of one or more metagenomic elements in Table 1 associated with health, wherein the subject is determined as having acne if one or more of the metagenomic elements in Table 1 associated with acne is enriched in the skin sample or one or more of the metagenomic elements in Table 1 associated with health is depleted in the skin sample.

In some embodiments, the microbial DNA is isolated from a skin follicle. Also provided herein are methods of determining whether a subject has acne, comprising obtaining a skin sample from a subject and analyzing (e.g., sequencing) the skin sample for enrichment or depletion of a metagenomic element disclosed herein. In some aspects, provided herein are methods of treating acne by administering a composition comprising a metabolite produced by a strain of P. granulosum.

Also provided here are methods for treating acne in a subject, comprising administering a composition comprising a metabolite produced by a strain of P. granulosum, P. avidum, and/or P. humerusii, wherein the metabolite is selected from bacterial culture supernatant, cell lysate, proteins, nucleic acids, lipids, and other bacterial molecules.

In some embodiments, the subject has a skin disease (e.g., inflammatory skin disease, such as acne, rosacea, or Porphyria Cutanea Tarda (PCT)).

Provided herein are compositions (e.g., cosmetic or pharmaceutical compositions) comprising at least one strain of P. granulosum, at least one strain of P. avidum, and/or at least one strain of P. humerusii to the subject. The strain of P. granulosum may be HL078PG1 and/or HL082PG1. The strain of P. avidum may be HL063PV1, HL083PV1, and HL307PV1. The strain of P. humerusii may be HL044PA1. The composition may further comprises one or more strains of P. acnes (e.g., an RT1, RT2, RT3, or RT6 strain of P. acnes). The composition may be enriched for any one of the genes associated with healthy skin in Table 1. In some embodiments, the composition comprises a phage against a strain of P. acnes (e.g., a strain of P. acnes selected from RT4, RT5, RT7, RT8, RT9 or RT10). In some embodiments, the composition comprises two or more phages against a strain of P. acnes. The composition may further comprise an antibiotic (preferably an antibiotic that does not target and/or kill P. granulosum, P. avidum, and/or P. humerusii). In certain embodiments, the compositions disclosed herein may be formulated for oral or topical delivery.

Also provided herein are methods of improving skin health or slowing or preventing skin aging in a subject, comprising administering a composition disclosed herein (e.g., a composition comprising P. granulosum, P. humerusii and/or P. avidum) to the subject.

### BRIEF DESCRIPTION OF THE FIGURES

FIG. 1 has four parts, A-D, and shows which bacteria dominate the skin follicular microbiome. Part A shows a box-and-whiskers plot comparing the relative abundances of bacteria, fungi, and P. acnes phage in the follicular microbiota of acne patients (n=38), age-matched healthy individuals (n=30), healthy individuals over 55 (H55+; n=4), and all healthy individuals combined (n=34). Part B shows a few fungal organisms were found in the follicle. Sequencing reads pooled from all subjects mapped to six fungal species, with less than 1× coverage for any species. Part C shows the relative abundance of P. acnes phage in all the samples suggests an increased prevalence and abundance of P. acnes phage in healthy individuals and a trend of increased phage abundance with age. Part D shows the relative abundances of bacterial species in the follicle. Each column represents the relative abundances of the bacterial species found in each individual. P. acnes was the dominant skin bacterium in all but one individual. On average P. acnes accounted for 91% of the bacterial taxa identified. An increase in the average relative abundances of P. acnes and P. granulosum was observed in the healthy individuals, whereas an increase in the average relative abundances of minor taxa was observed in the acne group. Five major skin bacterial species (P. acnes, P. humerusii, P. avidum, P. granulosum, and S. epidermidis) are shown separately from the phyla that they belong to.

FIG. 2 has three parts, A-C, which show the differences in the relative abundances of P. acnes operational gene units (OGUs) between acne patients and healthy individuals. Part A is a heat map showing the relative abundances of the OGUs in P. acnes loci 1, 2, and 3 in acne patients and healthy individuals. Each column represents an OGU, ordered based on the genomic location of the OGUs. OGUs 101-200 in the pan-genome were plotted to show locus 1, flanking OGUs, and locus 2. The 74 OGUs from locus 3, which is a plasmid, are also shown. Each row represents an individual. Acne patients (n=38) and healthy individuals (n=34, including those with age over 55) were compared. Individuals within each group were clustered based on the average relative abundance of locus 2 OGUs. Ribotype composition and past and current acne treatments are indicated on the right. Multiple treatments are depicted by more than one color. Part B shows fold changes in relative abundance of the OGUs in loci 1, 2, and 3 between acne patients and healthy individuals. Acne-associated OGUs had a fold change >1, while health-associated OGUs had a fold change <1. Part C shows prevalence ratio of the OGUs in loci 1, 2, and 3 between acne patients and healthy individuals. The presence of an OGU in a sample is defined as an OGU with at least 1× coverage after normalization.

FIG. 3 has two parts, A-B, which show the relative abundances of acne- and health-associated metagenomic elements in acne and healthy individuals. Part A shows the relative abundances of 62 P. acnes OGUs, including 25 acne- and 37 health-associated OGUs, and three organisms associated with healthy skin, P. acnes, P. granulosum, and P. acnes phage, were plotted for each individual to illustrate the importance of a balance between these metagenomic elements in health and acne. Each column represents an individual, and each row represents an OGU or an organism. The top ten ribotype composition and acne severity score (acne patients only) of each individual are also shown. Part B shows the prediction score of each individual based on the relative abundances of 45 metagenomic elements is shown, where red indicates acne and green indicates healthy skin. The classification of the clinical states had an overall accuracy of 85%.

FIG. 4 has two parts, A-B, which show class prediction accuracy using leave-one-out cross-validation and weighted gene-voting. Part A shows the training sample set (n=72), using the clinically defined acne and healthy individual grouping, the classifier correctly assigned the clinical states of 34 of the 49 assigned samples (69% accuracy) using a prediction strength threshold of 0.25. This result is statistically significant (P=0.001), because only one of the 1,000 permutated groupings had a higher accuracy. However, that particular grouping (accuracy of 72%) had fewer samples assigned than the clinical grouping (n=39 vs 49). This demonstrates that the differences in the relative abundances of the metagenomic elements between acne patients and healthy individuals can be used to predict the clinical states of the skin. When the refined set of 45 metagenomic elements was used, including 43 P. acnes OGUs, P. acnes locus 2, and P. granulosum, the prediction accuracy of the training sample set improved to 85%. Part B shows that the 45 metagenomic elements were able to assign 70% of the samples with 86% accuracy.

FIG. 5 shows histograms of age distribution within each subject group. Similar to previous studies, both teenagers and young adults were included in acne and healthy age-matched groups.

FIG. 6 shows P. acnes strain populations are different between acne patients and healthy individuals. Each column represents the relative abundances of the top ten P. acnes ribotypes in each individual. Acne patients, age-matched healthy individuals, and healthy individuals with age over 55 were included. Individuals often harbor more than one ribotype (an average of 2.3 ribotypes per person). Individuals were clustered based on the composition of the top ten ribotypes. Microbiome Types IV and V were found in the acne patients, but not in the healthy individuals.

FIG. 7 shows refaction curves indicate sufficient sequencing depth of all samples. The rarefaction curves of the 72 samples all reached the plateau, suggesting that the sequencing depth of all the samples was sufficient for detecting P. acnes OGUs and for comparative functional profiling. The sequencing depth ranged from 6.9×107-4.8×109 base pairs per sample. The rarefaction curves beyond 9×106 base pairs are not shown.

FIG. 8 shows functional profiles of the differentially abundant OGUs in acne patients and healthy individuals. P. acnes OGUs that were differentially abundant between acne patients and healthy individuals were assigned to functional categories. The functional profiles from the acne patients varied between individuals with higher abundances of unclassified genes, while in healthy individuals the functional profiles remained relatively stable across individuals.

FIG. 9 shows that Locus 2 encodes virulence-related genes. Locus 2 is a 20 Kb genomic island predominantly found in P. acnes clade IA-2 strains, RT4 and RT5. Locus 2 encodes 23 ORFs including a cluster of Streptolysin S-associated genes (sag) involved in biosynthesis and transport of bacterial toxins as well as self-immunity. Relative gene length and directionality for each gene encoded in locus 2 of P. acnes HL096PA1 is shown.

FIG. 10 shows that Locus 2 was more abundant in the acne patients with MTI than in the healthy individuals with MTI. The relative abundance of locus 2 in 15 acne patients and 13 healthy individuals with MTI is shown. Each column represents one of the 19 OGUs of locus 2, which were significantly different between acne patients and healthy individuals, plotted in the order of their genomic positions in the locus (as listed in Table 1). A significant increase (P=0.02) of the relative abundances of locus 2 OGUs was observed in acne patients compared to healthy individuals with MTI.

FIG. 11 shows that P. granulosum isolated from human facial skin produces very low levels of porphyrins, significantly lower than acne-associated P. acnes strains (RT4 strain HL053PA1 is shown). Supplementation with vitamin B12, shown previously to enhance porphyrin production in P. acnes, was unable to increase porphyrin production in P. granulosum. Each bar represents the porphyrins produced by each strain normalized to bacterial culture density. The means plus standard errors (error bars) of data obtained from at least three independent experiments with at least four replicates each are shown.

FIG. 12 shows strains of Propionibacterium granulosum, Propionibacterium avidum, and Propionibacterium humerusii produce much lower amounts of porphyrins than P. acnes. Each bar represents the porphyrins produced by each strain normalized by the bacterial culture density. Shown is the mean of the data obtained from at least four experiments with at least four replicates each for Propionibacterium granulosum (HL078PG1 and HL082PG1), Propionibacterium avidum (HL063PV1, HL083PV1, and HL307PV1), and Propionibacterium humerusii (HL044PA1) (grey bars). Some strains produced no or little detectable prophyrins as shown in the figure (close to zero). P. acnes HL053PA1 (RT4) is shown for comparison (black bar). Error bars represent standard error.

### DETAILED DESCRIPTION

In some aspects, provided herein are methods and compositions related to treating or preventing a skin disease (e.g., acne, rosacea, or PCT), preventing and/or slowing skin aging (e.g., preventing or inhibiting the formation of wrinkles), and maintaining healthy skin by administering to the subject a composition disclosed herein (e.g., a composition comprising at least one strain of P. granulosum, at least one strain of P. avidum, and/or at least one strain of P. humerusii). In some aspects, provided herein are methods of treating or preventing a skin condition in a subject and/or reducing the amount of porphyrins on the skin of a subject (e.g., a subject with symptoms of skin aging, or a subject with a skin condition, such as acne, rosacea, or PCT) by administering to the subject a composition comprising at least one strain of P. granulosum, at least one strain of P. avidum, and/or at least one strain of P. humerusii. In some aspects, the invention relates to determining whether a subject is at risk for a skin disease, comprising obtaining a skin sample from the subject, optionally isolating microbial DNA from the skin sample, and identifying the amount of P. granulosum, P. avidum, and/or P. humerusii in the microbiome of the skin sample. In some embodiments, the microbial DNA is isolated from a skin follicle. Also provided herein are methods of determining whether a subject has acne, comprising obtaining a skin sample from a subject and analyzing (e.g., sequencing) the skin sample for the enrichment or depletion of one or more metagenomic elements disclosed herein in the skin sample. In some aspects, provided herein are methods of treating acne comprising administering a composition comprising a metabolite produced by a strain of P. granulosum, P. avidum, and/or P. humerusii.

### Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “preventing” is art-recognized, and when used in relation to a condition, such as a local recurrence, is well understood in the art, and includes administration of a composition which reduces the frequency of, or delays the onset of, symptoms of a medical condition in a subject relative to a subject which does not receive the composition. Thus, prevention of acne includes, for example, reducing the number of detectable acne lesions in a population of patients receiving a prophylactic treatment relative to an untreated control population, and/or delaying the appearance of detectable lesions in a treated population versus an untreated control population, e.g., by a statistically and/or clinically significant amount.

The term “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

The term “ribotype” refers to strains of P. acnes. The ribotyped strains were characterized as in Fitz-Gibbon et al. (J. Investigative Dermatology 133:2152-60 (2013)).

The term “subject” refers to a mammal, including, but not limited to, a human or non-human mammal, such as a bovine, equine, canine, ovine, or feline.

A “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in a manner to improve or stabilize a subject's condition.

### Therapeutic Methods

Provided herein are methods of treating or preventing a skin condition, preventing and/or slowing skin aging, and/or maintaining healthy skin. In some aspects, the methods relate to treating or preventing a skin disease in a subject, comprising administering a composition comprising Propionibacterium granulosum (P. granulosum), P. avidum, and/or P. humerusii to the subject. Compositions described herein may have at least one strain of P. granulosum, at least one strain of P. avidum, and/or at least one strain of P. humerusii. Compositions may contain two or more, three or more, four or more, five or more, six or more, seven or more, eight or more, nine or more, or ten or more strains of P. granulosum, P. avidum, and/or P. humerusii. In some aspects, provided herein are methods related to reducing the amount of porphyrins on the skin of a subject, comprising administering a composition comprising P. granulosum, P. avidum, and/or P. humerusii. In some embodiments, the subject has a skin disease or symptoms associated with skin aging (e.g., formation of wrinkles). In some embodiments, the skin disease is an inflammatory skin disease, such as acne, rosacea, or Porphyria Cutanea Tarda (PCT). The composition may further comprise an additional strain of bacteria (e.g., a strain of P. acnes, such as RT1, RT2, RT3, or RT6 strain of P. acnes). In some aspects, provided herein are methods of slowing or preventing skin aging in a subject by administering a composition comprising P. granulosum, P. avidum, and/or P. humerusii to the subject.

The strain of P. granulosum may be HL078PG1 and/or HL082PG1. The strain of P. avidum may be HL063PV1, HL083PV1, and HL307PV1. The strain of P. humerusii may be HL044PA1.

In some embodiments, the composition further comprises a phage against a strain of P. acnes (e.g., a RT4, RT5, RT7, RT8, RT9 or RT10 strain of P. acnes). In some embodiments, the composition comprises two or more (e.g., three or more, four or more, five or more, six or more, seven or more, eight or more, nine or more, or ten or more) phages against a strain of P. acnes. The type of phage that may be administered in a composition disclosed herein depends on the type of acne or skin disease, the medical history of an individual, or the symptoms of a subject with a skin disease. Non-limiting examples of phages include PHL111M01, PHL082M00, PHL060L00, PHL067M10, PHL071N05, PHL112N00, PHL037M02, PHL085N00, PHL115M02, PHL085M01, PHL114L00, PHL010M04, PHL066M04, PHL071N05, PHL113M01, PHL112N00, and PHL037M02. Information about P. acnes phages can be found in U.S. Patent Publication US20150086581A1, hereby incorporated in its entirety. In some embodiments, the composition may comprise an antibiotic (e.g., an antibiotic that does not target P. granulosum, P. avidum, and/or P. humerusii).

The compositions disclosed herein may be administered to a subject by any means known in the art, for example, the composition may be formulated for topical delivery. The formulation may be a liquid, gel, or cream. In some embodiments, the composition is formulated for oral delivery. The composition may be in the form of a pill, tablet, or capsule. In some embodiments, the subject may be a mammal (e.g., a human). In some embodiments, the composition is self-administered.

In some aspects, provided herein are methods of determining whether a subject is at risk for a skin disease by obtaining a skin sample from the subject, isolating bacterial DNA from the skin sample, identifying the amount of P. granulosum in the microbiome of the skin sample, and if P. granulosum comprises less than 5%, less than 4.5%, less than 4%, less than 3.5%, less than 3%, less than 2.5%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, less than 0.01% of the microbiome of the skin sample, the subject is considered at risk for a skin disease. In some embodiments, the composition comprising P. granulosum is administered to the subject if P. granulosum comprises less than 5%, less than 4.5%, less than 4%, less than 3.5%, less than 3%, less than 2.5%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, less than 0.01% of the microbiome of the skin sample.

In some aspects, provided herein are methods of determining whether a subject is at risk for a skin disease by obtaining a skin sample from the subject, isolating bacterial DNA from the skin sample, identifying the amount of P. avidum in the microbiome of the skin sample, and if P. avidum comprises less than 5%, less than 4.5%, less than 4%, less than 3.5%, less than 3%, less than 2.5%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, less than 0.01% of the microbiome of the skin sample, the subject is considered at risk for a skin disease. In some embodiments, the composition comprising P. avidum is administered to the subject if P. avidum comprises less than 5%, less than 4.5%, less than 4%, less than 3.5%, less than 3%, less than 2.5%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, less than 0.01% of the microbiome of the skin sample.

In some aspects, provided herein are methods of determining whether a subject is at risk for a skin disease by obtaining a skin sample from the subject, isolating bacterial DNA from the skin sample, identifying the amount of P. humerusii in the microbiome of the skin sample, and if P. humerusii comprises less than 5%, less than 4.5%, less than 4%, less than 3.5%, less than 3%, less than 2.5%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, less than 0.01% of the microbiome of the skin sample, the subject is considered at risk for a skin disease. In some embodiments, the composition comprising P. humerusii is administered to the subject if P. humerusii comprises less than 5%, less than 4.5%, less than 4%, less than 3.5%, less than 3%, less than 2.5%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, less than 0.01% of the microbiome of the skin sample. In some aspects, provided herein are methods of determining whether a subject has a skin disease (e.g., acne), by obtaining a skin sample from the subject, sequencing DNA from the skin sample, analyzing the DNA for the enrichment of a metagenomic element in Table 1 associated with acne or the depletion of a metagenomic element in Table 1 associated with healthy skin, and determining the subject to have a skin disease (e.g., acne) if the metagenomic element in Table 1 associated with acne is enriched in the skin sample or if one or more of the metagenomic elements in Table 1 associated with health (e.g., healthy skin, or skin not afflicted with a skin disease, such as acne) is depleted in the skin sample. The DNA in the skin sample may be sequenced by any known technique in the art, including, but not limited to, Maxam Gilbert sequencing, Sanger sequencing, shotgun sequencing, bridge PCR, or next generation sequencing methods, such as massively parallel signature sequencing (MPSS), polony sequencing, 454 pyrosequencing, Illumina (Solexa) sequencing, SOLiD sequencing, Ion torrent semiconductor sequencing, DNA nanoball sequencing, heliscope single molecule sequencing, single molecule real time (SMRT) sequencing, or nanopore DNA sequencing. In some embodiments, the skin sample is considered enriched for a metagenomic elements in Table 1 associated with acne afflicted skin if at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, or at least 100% of the bacteria in the sample show enrichment of the metagenomic element in Table 1 associated with acne, or if the level of a metagenomic element in Table 1 across the sample is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, or at least 100% higher than a level associated with health. Similarly, the skin sample may be considered depleted for a metagenomic element in Table 1 associated with healthy skin if less than 100%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5% of the bacteria in the sample show depletion of the metagenomic element in Table 1 associated with healthy skin, or if the level of a metagenomic element in Table 1 across the sample is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 95% lower than a level associated with health.

In some aspects, provided herein are methods for determining whether a subject has a skin disease (e.g., acne) comprising obtaining a skin sample from the subject, isolating microbial DNA from the skin sample, using one or more primer sets to amplify the DNA, using one or more probes to detect the amplified DNA, and analyzing the probe signals for the enrichment of one or more metagenomic elements in Table 1 associated with acne or the depletion of one or more metagenomic elements in Table 1 associated with health (e.g., healthy skin, or skin not afflicted with a skin disease, such as acne), wherein the subject is determined as having acne if one or more of the metagenomic elements in Table 1 associated with acne is enriched in the skin sample or if one or more of the metagenomic elements in Table 1 associated with health is depleted in the skin sample.

In some embodiments, the skin sample is enriched if at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, or at least 100% of the bacteria in the sample has the metagenomic element in Table 1 associated with acne, or if the level of a metagenomic element in Table 1 across the sample is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, or at least 100% higher than a level associated with health. In some embodiments, the skin sample is depleted of a metagenomic element if less than 100%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5% of the bacteria in the sample has a metagenomic element in Table 1 associated with healthy skin, or if the level of a metagenomic element in Table 1 across the sample is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 95% lower than a level associated with health.

TABLE 1
Exemplary P. acnes OGUs Associated with Health or Acne

| | | Clinical |
|---|---|---|
| P. acnes Gene ID | Predicted function | Association |
| PAGK_0029 | Transcriptional regulator | Acne |
| PAGK_0033 | Acetyltransferase | Health |
| PAGK_0136 | Putative glycosyl transferase | Health |
| PAGK_0138 | Chain-length determining protein | Health |
| PAGK_0160 (Locus 2) | Hypothetical protein | Acne |
| PAGK_0161 (Locus 2) | Hypothetical protein | Acne |
| PAGK_0162 (Locus 2) | Single-strand binding protein (Ssb) | Acne |
| PAGK_0163 (Locus 2) | Cobalamin biosynthesis protein CobQ/partitioning | Acne |
| PAGK_0164 (Locus 2) | Yag1E | Acne |
| PAGK_0165 (Locus 2) | Putative replication protein | Acne |
| PAGK_0166 (Locus 2) | Site-specific integrase-resolvase | Acne |
| PAGK_0167 (Locus 2) | Hypothetical protein | Acne |
| PAGK_0168 (Locus 2) | Hypothetical protein | Acne |
| PAGK_0169 (Locus 2) | ATPase/chromosome partitioning | Acne |
| PAGK_0170 (Locus 2) | Caax amino terminal protease | Acne |
| PAGK_0171 (Locus 2) | Abi domain protein | Acne |
| PAGK_0172 (Locus 2) | YcaO-like family protein (SagD-like) | Acne |
| PAGK_0173 (Locus 2) | Cyclodehydratase protein (SagC-like) | Acne |
| PAGK_0174 (Locus 2) | Oxidoreductase protein (SagB-like) | Acne |
| PAGK_0029 | Transcriptional regulator | Acne |
| PAGK_0033 | Acetyltransferase | Health |
| PAGK_0175 (Locus 2) | EXLDI domain protein | Acne |
| PAGK_0176 (Locus 2) | ABC transporter/ATP-binding protein | Acne |
| PAGK_0177 (Locus 2) | ABC-2 type membrane transporter | Acne |
| PAGK_0178 (Locus 2) | Hypothetical protein | Acne |
| PAGK_0198 | ABC transporter | Acne |
| PAGK_0374 | 2-C-methyl-D-erythritol 4-phosphate | Health |
| PAGK_0387 | L-asparaginase I | Acne |
| PAGK_0505 | Sugar transporter family protein | Health |
| PAGK_0665 | Glutamate dehydrogenase | Health |
| PAGK_0667 | Putative 6-aminohexanoate-dimer hydrolase | Health |
| PAGK_0687 | Hypothetical protein | Health |
| PAGK_0712 | Hypothetical protein | Health |
| PAGK_0754 | DNA processing/uptake protein | Health |
| PAGK_0755 | Methylated-DNA--protein-cysteine | Health |
| PAGK_0768 | Translation initiation factor IF-3 | Health |
| PAGK_0793 | Site-specific tyrosine recombinase XerD | Health |
| PAGK_0820 | Putative acyltransferase | Health |
| PAGK_0821 | D-alanine--D-alanine ligase | Health |
| PAGK_0880 | Putative ATP-binding ABC transporter protein | Health |
| PAGK_0896 | Diaminopimelate decarboxylase | Health |
| PAGK_0979 | Hypothetical protein | Health |
| PAGK_0983 | Hypothetical protein | Health |
| PAGK_0985 | Metallo-beta-lactamase superfamily protein | Health |
| PAGK_1034 | Hypothetical protein | Health |
| PAGK_1035 | Cobalamin independent methionine synthase | Health |
| PAGK_1039 | Putative alpha-amylase | Health |
| PAGK_1043 | Putative pyrophosphohydrolase | Health |
| PAGK_1113 | Putative aminooxidase | Health |
| PAGK_1168 | 2-dehydropantoate 2-reductase | Health |
| PAGK_1302 | GTPase ObgE | Health |
| PAGK_1393 | Putative helicase protein | Health |
| PAGK_1606 | Ferrous iron transport protein B | Health |
| PAGK_1635 | Hypothetical protein | Health |
| PAGK_1813 | Exodeoxyribonuclease III | Health |
| PAGK_1815 | Hypothetical protein | Health |
| PAGK_1820 | 2-oxoglutarate ferredoxin oxidoreductase | Health |
| PAGK_2086 | Potassium transporter | Health |
| PAGK_2104 | Thiazolylpeptide-type bacteriocin precursor | Acne |
| PAGK_2105 | ABC transporter/ATP-binding protein | Acne |
| PAGK_2133 | Single-strand DNA-binding protein | Health |
| PAGK_2214 | Glycerol uptake facilitator protein | Health |
| PAGK_2332 (Locus 3) | Plasmid partitioning protein ParA | Acne |
| PAGK_0029 | Transcriptional regulator | Acne |
| PAGK_0033 | Acetyltransferase | Health |
| HMPREF9572DFT_Contig29. | Hypothetical protein | Health |
| 2.Glimmer3.p5_hybrid.274 | | |

The skin sample may be any sample taken from the skin of a subject (e.g., a human). The subject may have skin afflicted with an inflammatory skin disease (e.g., acne, PCT, or rosacea) or may have skin not afflicted with a skin disease. A skin sample may be obtained by any means known in the art including, but not limited to, swabbing the skin with a tool able to collect skin cells (e.g., a Q-tip or cotton swab), placing an adhesive or tape on the surface of the skin and removing the adhesive or tape, thereby yielding a skin sample on the adhesive or tape, or through a biopsy (e.g., a shave biopsy, a punch biopsy, an incisional biopsy, a saucerization biopsy, or an excisional biopsy).

In some embodiments, the skin sample includes epithelial cells, epidermal cells, dermal cells, skin flora (e.g., skin microbes such as P. granulosum, P. humerusii, P. avidum, P. acnes, Staphylococcus epidermidis, Staphylococcus aureus, Streptococcus pyogenes, Streptococcus mitis, Propionibacterium acnes, Corynebacterium spp., Acinetobacter johnsonii, Pseudomonas aeruginosa), fungus (e.g., Candida albicans, Rhodotorula rubra, Torulopsis and Trichosporon cutaneum, dermatophytes (skin living fungi) such as Microsporum gypseum, and Trichophyton rubrum and nondermatophyte fungi (opportunistic fungi that can live in skin) such as Rhizopus stolonifer, Trichosporon cutaneum, Fusarium, Scopulariopsis brevicaulis, Curvularia, Alternaria alternata, Paecilomyces, Aspergillus flavus and Penicillium species), hair/skin follicles, adipose tissue (i.e., subcutaneous fat), and/or connective tissue. Preferably the skin sample includes at least some skin cells of the patient (whether living or dead) and skin flora.

In some aspects, provided herein are methods for treating acne in a subject, comprising administering a composition comprising a metabolite produced by a strain of P. granulosum, P. humerusii, and/or P. avidum wherein the metabolite is selected from bacterial culture supernatant, cell lysate, proteins, nucleic acids, lipids, and other bacterial molecules.

In general, the above methods directly act to reduce the amount of pathogenic bacteria in a subject. In some embodiments, this includes any such therapy that achieves the same goal of reducing the number of pathogenic organisms, when used in combination with the composition described herein, would lead to replacement of the pathogenic microflora involved in the diseased state with natural microflora enriched in skin not afflicted with a skin disease, or less pathogenic species occupying the same ecological niche as the type causing a disease state. For example, a subject may undergo treatment with antibiotics or a composition comprising antibiotics to target and decrease the prevalence of pathogenic organisms, and subsequently be treated with a composition described herein.

Suitable antimicrobial compounds include capreomycins, including capreomycin IA, capreomycin IB, capreomycin IIA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefmenoxime, cefmetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cephalosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dirithromycin, doxycyclin, epicillin, erythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin A1, lincomycin, lomefloxacin, loracarbef, lymecycline, meropenam, metampicillin, methacycline, methicillin, mezlocillin, micronomicin, midecamycins such as midecamycin A1, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin O, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and analogs, derivatives, pharmaceutically acceptable salts, esters, prodrugs, and protected forms thereof.

Suitable anti-fungal compounds include ketoconazole, miconazole, fluconazole, clotrimazole, undecylenic acid, sertaconazole, terbinafine, butenafine, clioquinol, haloprogin, nystatin, naftifine, tolnaftate, ciclopirox, amphotericin B, or tea tree oil and analogs, derivatives, pharmaceutically acceptable salts, esters, prodrugs, and protected forms thereof.

Suitable antiviral agents include acyclovir, azidouridine, anismoycin, amantadine, bromovinyldeoxusidine, chlorovinyldeoxusidine, cytarabine, delavirdine, didanosine, deoxynojirimycin, dideoxycytidine, dideoxyinosine, dideoxynucleoside, desciclovir, deoxyacyclovir, efavirenz, enviroxime, fiacitabine, foscamet, fialuridine, fluorothymidine, floxuridine, ganciclovir, hypericin, idoxuridine, interferon, interleukin, isethionate, nevirapine, pentamidine, ribavirin, rimantadine, stavudine, sargramostin, suramin, trichosanthin, tribromothymidine, trichlorothymidine, trifluorothymidine, trisodium phosphomonoformate, vidarabine, zidoviridine, zalcitabine and 3-azido-3-deoxythymidine and analogs, derivatives, pharmaceutically acceptable salts, esters, prodrugs, and protected forms thereof.

Other suitable antiviral agents include 2′,3′-dideoxyadenosine (ddA), 2′,3′-dideoxyguanosine (ddG), 2′,3′-dideoxycytidine (ddC), 2′,3′-dideoxythymidine (ddT), 2′3′-dideoxy-dideoxythymidine (d4T), 2′-deoxy-3′-thia-cytosine (3TC or lamivudime), 2′,3′-dideoxy-2′-fluoroadenosine, 2′,3′-dideoxy-2′-fluoroinosine, 2′,3′-dideoxy-2′-fluorothymidine, 2′,3′-dideoxy-2′-fluorocytosine, 2′3′-dideoxy-2′,3′-didehydro-2′-fluorothymidine (Fd4T), 2′3′-dideoxy-2′-beta-fluoroadenosine (F-ddA), 2′3′-dideoxy-2′-beta-fluoro-inosine (F-ddI), and 2′,3′-dideoxy-2′-beta-flurocytosine (F-ddC). In some embodiments, the antiviral agent is selected from trisodium phosphomonoformate, ganciclovir, trifluorothymidine, acyclovir, 3′-azido-3′-thymidine (AZT), dideoxyinosine (ddI), and idoxuridine and analogs, derivatives, pharmaceutically acceptable salts, esters, prodrugs, and protected forms thereof.

### Pharmaceutical Compositions

In some aspects, the invention relates to a composition (e.g., a pharmaceutical composition) comprising a strain of P. granulosum, a strain of P. humerusii, or a strain of P. avidum. A composition may have two or more, three or more, four or more, five or more, six or more, seven or more, eight or more, nine or more, or ten or more strains of P. granulosum, P. humerusii, and/or P. avidum. A composition may comprise multiple strains from one species of bacteria, or a composition may have multiple strains from different species of bacteria. For example, a composition disclosed herein may have multiple strains of P. granulosum, or may have a combination of strains from P. granulosum, P. avidum, and/or P. humerusii. The pharmaceutical composition may be formulated for topical administration. The pharmaceutical composition may be a probiotic. The pharmaceutical compositions disclosed herein may be delivered by any suitable route of administration, including orally, buccally, sublingually, parenterally, and topically, as by powders, ointments, drops, liquids, gels, or creams. In certain embodiments, the pharmaceutical compositions are delivered generally (e.g., via oral or parenteral administration). In certain other embodiments, the pharmaceutical compositions are delivered locally through injection.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

Exemplary identities of various constituents of the topical formulations of some embodiments of the present invention are described below.

### Vehicles

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol, dimethicone, and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In some embodiments, formulations do not have methanol, ethanol, propanols, or butanol.

### Surfactants and Emulsifiers

Many topical formulations contain chemical emulsions which use surface active ingredients (emulsifiers and surfactants) to disperse dissimilar chemicals in a particular solvent system. For example, most lipid-like (oily or fatty) or lipophilic ingredients do not uniformly disperse in aqueous solvents unless they are first combined with emulsifiers, which form microscopic aqueous soluble structures (droplets) that contain a lipophilic interior and a hydrophilic exterior, resulting in an oil-in-water emulsion. In order to be soluble in aqueous media, a molecule must be polar or charged so as to favorably interact with water molecules, which are also polar. Similarly, to dissolve an aqueous-soluble polar or charged ingredient in a largely lipid or oil-based solvent, an emulsifier is typically used which forms stable structures that contain the hydrophilic components in the interior of the structure while the exterior is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion droplet. Emulsion destabilization results in the aqueous and lipophilic ingredients separating into two layers, potentially destroying the commercial value of a topical product.

Surfactants suitable for use in the present invention may be ionic or non-ionic. These include, but are not limited to: cetyl alcohol, polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-10 phosphate, methylbenzethonium chloride, dicetyl phosphate, ceteth-10 phosphate (ceteth-10 is the polyethylene glycol ether of cetyl alcohol where n has an average value of 10; ceteth-10 phosphate is a mixture of phosphoric acid esters of ceteth-10), ceteth-20, Brij S10 (polyethylene glycol octadecyl ether, average Mₙ˜711), and Poloxamers (including, but not limited to, Poloxamer 188 (HO(C₂H₄O)ₐ(CH(CH₃)CH₂O)_{b}(C₂H₄O)ₐH, average molecular weight 8400) and Poloxamer 407 (HO(C₂H₄O)ₐ(CH(CH₃)CH₂O)_{b}(C₂H₄O)ₐH, wherein a is about 101 and b is about 56)). Appropriate combinations or mixtures of such surfactants may also be used according to the present invention. Many of these surfactants may also serve as emulsifiers in formulations of the present invention.

Other suitable emulsifiers for use in the formulations of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol (cetearyl alcohol), ceteareth-12, ceteareth-20, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate, PEG-75 stearate, PEG-100 stearate, and PEG-100 stearate, ceramide 2, ceramide 3, stearic acid, cholesterol, steareth-2, and steareth-20, or combinations/mixtures thereof, as well as cationic emulsifiers like stearamidopropyl dimethylamine and behentrimonium methosulfate, or combinations/mixtures thereof.

### Moisturizers, Emollients, and Humectants

One of the most important aspects of topical products in general, and cosmetic products in particular, is the consumer's perception of the aesthetic qualities of a product. For example, while white petrolatum is an excellent moisturizer and skin protectant, it is rarely used alone, especially on the face, because it is greasy, sticky, does not rub easily into the skin and may soil clothing. Consumers highly value products which are aesthetically elegant and have an acceptable tactile feel and performance on their skin. Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800. Suitable emollients or humectants for use in the formulations of the present invention include, but are not limited to, panthenol, cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, isononyl isononanoate, and 1,3-bis(N-2-(hydroxyethyl)palmitoylamino)-2-hydroxypropane. In addition, appropriate combinations and mixtures of any of these moisturizing agents and emollients may be used in accordance with the present invention.

### Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols, tocopheryl acetate, sodium ascorbate/ascorbic acid, ascorbyl palmitate, propyl gallate, and chelating agents like EDTA (e.g., disodium EDTA), citric acid, and sodium citrate.

In some embodiments, the antioxidant or preservative comprises (3-(4-chlorophenoxy)-2-hydroxypropyl)carbamate.

In some embodiments, antioxidants or preservatives of the present invention may also function as a moisturizer or emollient, for example.

In addition, combinations or mixtures of these preservatives or anti-oxidants may also be used in the formulations of the present invention.

### Combination Agents

The composition can also contain any other agent that has a desired effect when applied topically to a subject. Suitable classes of active agents include, but are not limited to antibiotic agents (i.e., antibiotic agents that dot not target P. granulosum), antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones. Mixtures of any of these active agents may also be employed. Additionally, dermatologically-acceptable salts and esters of any of these agents may be employed.

### Viscosity Modifiers

Suitable viscosity adjusting agents (i.e., thickening and thinning agents or viscosity modifying agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. In addition, appropriate combinations or mixtures of these viscosity adjusters may be utilized according to the present invention.

### Additional Constituents

Additional constituents suitable for incorporation into the emulsions of the present invention include, but are not limited to: skin protectants, adsorbents, demulcents, emollients, moisturizers, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, lipids, immunomodulators, and pH adjusters (e.g., citric acid, sodium hydroxide, and sodium phosphate). For example, lipids normally found in healthy skin (or their functional equivalents) may be incorporated into the emulsions of the present invention. In certain embodiments, the lipid is selected from the group consisting of ceramides, cholesterol, and free fatty acids. Examples of lipids include, but are not limited to, ceramide 1, ceramide 2, ceramide 3, ceramide 4, ceramide 5, ceramide 6, hydroxypropyl bispalmitamide MEA, and hydroxypropyl bislauramide MEA, and combinations thereof.

Examples of peptides that interact with protein structures of the dermal-epidermal junction include palmitoyl dipeptide-5 diaminobutyloyl hydroxythreonine and palmitoyl dipeptide-6 diaminohydroxybutyrate.

Examples of skin soothing agents include, but are not limited to algae extract, mugwort extract, stearyl glycyrrhetinate, bisabolol, allantoin, aloe, avocado oil, green tea extract, hops extract, chamomile extract, colloidal oatmeal, calamine, cucumber extract, and combinations thereof.

In certain embodiments, the compositions comprise bergamot or bergamot oil. Bergamot oil is a natural skin toner and detoxifier. In certain embodiments, it may prevent premature aging of skin and may have excellent effects on oily skin conditions and acne.

In some embodiments, the composition comprises a vitamin. Examples of vitamins include, but are not limited to, vitamins A, D, E, K, and combinations thereof. Vitamin analogues are also contemplated; for example, the vitamin D analogues calcipotriene or calcipotriol. In some embodiments, the vitamin may be present as tetrahexyldecyl ascorbate. This compound exhibits anti-oxidant activity, inhibiting lipid peroxidation. In certain embodiments, use can mitigate the damaging effects of UV exposure. Studies have shown it to stimulate collagen production as well as clarifying and brightening the skin by inhibiting melanogenesis (the production of pigment) thereby promoting a more even skin tone.

In some embodiments, the composition comprises a sunscreen. Examples of sunscreens include, but are not limited to, p-aminobenzoic acid, avobenzone, cinoxate, dioxybenzone, homosalate, menthyl anthranilate, octocrylene, octyl methoxycinnamate, octyl salicylate, oxybenzone, padimate 0, phenylbenzimidazole sulfonic acid, sulisobenzone, titanium dioxide, trolamine salicylate, zinc oxide, 4-methylbenzylidene camphor, methylene bis-benzotriazolyl tetramethylbutylphenol, bis-ethylhexyloxyphenol methoxyphenyl triazine, terephthalylidene dicamphor sulfonic acid, drometrizole trisiloxane, disodium phenyl dibenzimidazole tetrasulfonate, diethylamino hydroxybenzoyl hexyl benzoate, octyl triazone, diethylhexyl butamido triazone, polysilicone-15, and combinations thereof.

Suitable fragrances and colors may be used in the formulations of the present invention. Examples of fragrances and colors suitable for use in topical products are known in the art.

### EXEMPLIFICATION

### Example 1: High Abundance of Propionibacteria in the Follicular Microbiota

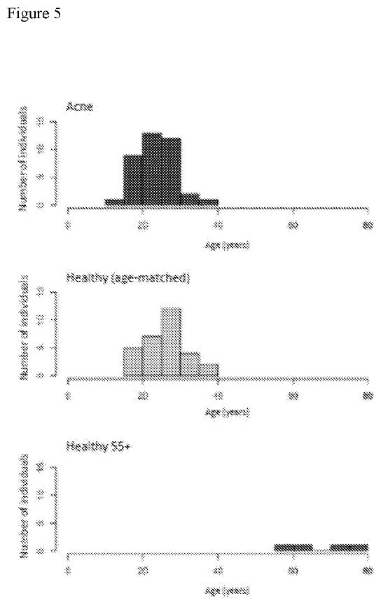

To identify the disease- and health-associated microbial elements in the follicular microbiota, ultra-deep metagenomic shotgun sequencing was performed of the samples collected from 38 acne patients and 30 age-matched healthy individuals (FIG. 5). Below is a Table which shows clinical information of the acne patients (n=38), age-matched healthy individuals (n=30), and healthy individual with age over 55 (n=4).

| | | Healthy | |
|---|---|---|---|
| Clinical Group | Acne | (age-matched) | Healthy 55+ |
| Number of subjects | 38 | 30 | 4 |

Gender n (%)

| | | | | |
|---|---|---|---|---|
| Males | 13 | (34%) | 16 (53%) | 2 (50%) |
| Females | 25 | (66%) | 14 (47%) | 2 (50%) |

Age (years)

| | | | |
|---|---|---|---|
| Range | 14-37 | 16-38 | 55-79 |
| Average | 24 | 26 | 67.5 |
| Median | 24.5 | 27 | 68 |

Ethnicity/Race n (%)

| | | | | |
|---|---|---|---|---|
| American Indian | 1 | (2.6%) | 0 | 0 |
| Asian | 12 | (31.6%) | 10 (33%) | 2 (50%) |
| Black | 6 | (15.8%) | 0 | 0 |
| Hispanic | 5 | (13%) | 10 (33%) | 0 |
| White | 11 | (29%) | 8 (27%) | 2 (50%) |
| More than one race | 3 | (8%) | 2 (7%) | 0 |

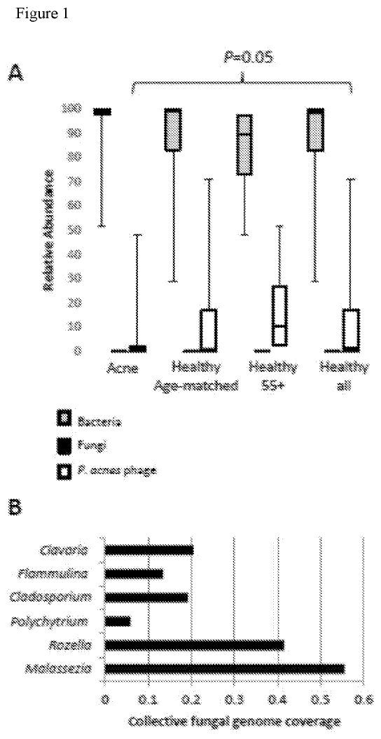

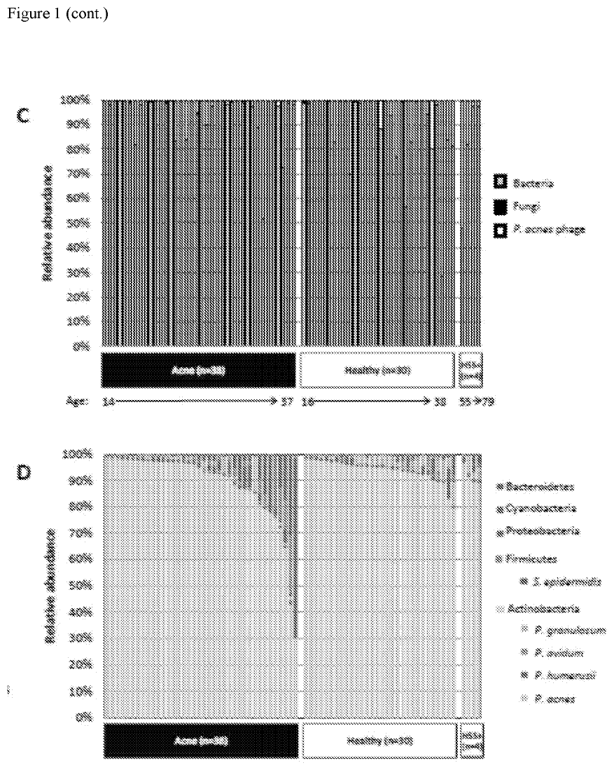

FIG. 5 shows removal of human DNA sequences and low-quality reads, an average of 1.08 gigabase pairs (Gbp) per sample (6.9×10⁷ bp-4.8×10⁹ bp) was obtained, sufficient to cover the microbial diversity of skin samples. The cleaned sequencing reads were mapped to the reference genome set, which consists of 1,252 bacterial and 272 fungal genomes from the HMP reference genome database and several additional genomes of skin microorganisms: Propionibacterium avidum, Propionibacterium granulosum, Propionibacterium humerusii, and P. acnes bacteriophage. To cover different P. acnes strains, the P. acnes pan-genome was also included in the reference. Consistent with other skin sites, bacteria were the major organisms found in the follicle (FIG. 1, Part A) with few fungal organisms detected at low relative abundances (FIG. 1, Part B, Part C). Five main bacterial phyla were found in the samples, including Actinobacteria (95.6%), Firmicutes (2.3%), Proteobacteria (1.2%), Cyanobacteria (0.6%), and Bacteroidetes (0.2%). The dominance of Actinobacteria in the follicle is consistent with previous taxonomic analyses of sebaceous skin sites.

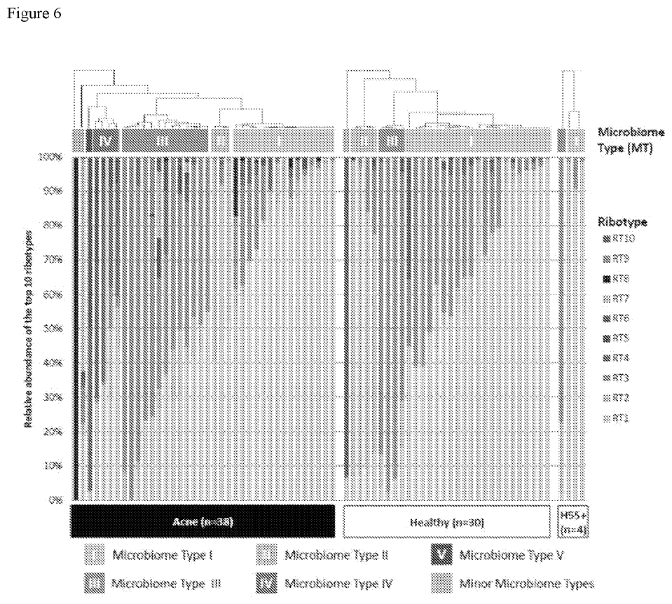

At the species level, P. acnes was the most prevalent and abundant. It was found in all 68 individuals with an average relative abundance of 91.0% (FIG. 1, Part D; Table 2). The healthy individuals had a slightly higher relative abundance of P. acnes than the acne patients (93.8% vs. 88.5%). The differences in P. acnes strain populations between acne patients and healthy individuals were compared. The relative abundances of the dominant P. acnes strains were assigned based on the read coverages of the 16S rRNA SNPs that distinguish the ten most common (top ten) P. acnes ribotypes (RTs) (FIG. 6). It was found that acne patients had a higher diversity of P. acnes population (Shannon index=1.07) with more RTs (2.55 RTs per individual) than healthy individuals (Shannon index=0.79, P=0.039; 1.97 RTs per individual, P=0.007). Consistent with the previous study, RT4, RT5, and RT8 were found to be more abundant and more prevalent in acne patients, with RT4 being statistically significantly different in relative abundance (P=0.004), while RT2 and RT6 were more abundant in healthy individuals (FIG. 6). The strain association with acne or healthy skin is consistent with previous multi-locus sequence typing and 16S ribotype analysis of large collections of clinical P. acnes isolates.

TABLE 2
Bacterial organisms identified in the follicular microbiome of the three groups.

| | | | | Healthy | | | | | |
|---|---|---|---|---|---|---|---|---|---|
| | | Acne | | age-matched | | Healthy 55+ | | Acne | |
| | | (n = 38) | | (n = 30) | | (n = 4) | | vs. age- | Acne |
| | | Average | | Average | | Average | | matched | vs. all |
| | Ref. | relative | Prevalence | relative | Prevalence | relative | Prevalence | healthy | healthy |
| Phylum | species | abundance | (%) | abundance | (%) | abundance | (%) | P-value | P-value |
| Actinobacteria | P. | 88.50% | 38 (100%) | 93.80% | 30 (100%) | 91.60% | 4 (100%) | 0.663 | 0.810 |
| | acnes | | | | | | | | |
| | P. | 3.60% | 21 (55%) | 1.66% | 22 (73%) | 1.40% | 2 (50%) | 0.654 | 0.586 |
| | humerusii | | | | | | | | |
| | P. | 0.90% | 9 (24%) | 2.19% | 17 (57%) | 2.12% | 3 (75%) | 0.002 | 0.001 |
| | granulosum | | | | | | | | |
| | P. | 0.37% | 6 (16%) | 0.43% | 9 (30%) | 0.55% | 0 | 0.859 | 0.469 |
| | avidum | | | | | | | | |
| | Other | 0.08% | — | 0.07% | — | 0.24% | — | — | — |
| Firmicutes | S. | 1.01% | 9 (24%) | 0.64% | 6 (20%) | 0.95% | 1 (25%) | 0.568 | 0.666 |
| | epidermidis | | | | | | | | |
| | Other | 2.34% | — | 0.48% | — | 0.46% | — | — | — |

| | | | | | | | | |
|---|---|---|---|---|---|---|---|---|
| Proteobacteria | 1.88% | — | 0.36% | — | 1.83% | — | — | — |
| Cyanobacteria | 1.02% | — | 0.23% | — | 0% | — | — | — |
| Bacteroidetes | 0.26% | — | 0.07% | — | 0% | — | — | — |

Other cutaneous Propionibacterium species, including P. humerusii (2.7%), P. granulosum (1.6%), and P. avidum (0.4%), were also detected (Table 2). Among them, P. granulosum, a resident bacterium of sebaceous skin sites, was significantly more abundant in the healthy individuals than in the acne patients (P=0.002). This is the same trend as seen in P. acnes.

Other bacterial species frequently identified in the follicle microbiota include Staphylococcus epidermidis (0.9%), Staphylococcus capitis (0.4%), Escherichia coli (0.7%), and Clostridium sp. (0.5%). Minor bacterial taxa were found more prevalent and abundant in acne patients than in healthy individuals with varying presence and abundance. On average, acne patients had a higher diversity at the species level than healthy individuals, but not significantly different (Shannon index=0.63 and 0.44, respectively; P=0.072). Reduced P. acnes and P. granulosum and increased minor taxa observed in acne patients suggest disruptions of the commensal skin flora in acne development.

### Example 2: The Skin Microbiome of Older Healthy Individuals is Similar to Younger Healthy Individuals

Acne rarely occurs in individuals over the age of 50. The skin microbiome profiles of older individuals with clear skin can be used as independent references for healthy, acne-free skin. However, the skin metagenome of older healthy individuals has not been characterized. Metagenomic shotgun sequencing was performed of the samples collected from four healthy individuals of age 55-79 (FIG. 6). The bacterial compositions of the follicular microbiome of these individuals were similar to those of the younger healthy individuals (FIG. 1, Part D). It was previously noted that the production of sebum, a nutrient source for lipophilic bacteria, including Propionibacterium species, decreases with age. In the analysis, the relative abundances of both P. acnes and P. granulosum were higher in older healthy individuals than in acne patients (Table 1), similar to the observations seen in younger healthy individuals. When grouping all healthy individuals together, P. granulosum remained significantly more abundant in healthy skin than acne (P=0.001). At the strain level, P. acnes populations in older healthy individuals were similar to those of the younger healthy cohort, consisting of mostly RT1, RT2, and RT3 strains, with little or no RT4, RT5, and RT8 strains detected (FIG. 6).

When comparing all 34 healthy individuals, including the four subjects over the age of 55, with the 38 acne patients, were found an increased prevalence and abundance of P. acnes phage in the healthy group (P=0.05) (FIG. 1, Part A). A trend between age and the relative abundance of P. acnes phage was observed. Higher relative abundances of phage were found more often in individuals with increased age (FIG. 1, Part C).

### Example 3: Metagenomic Composition of the Skin Microbiome is Different Between Acne Patients and Healthy Individuals

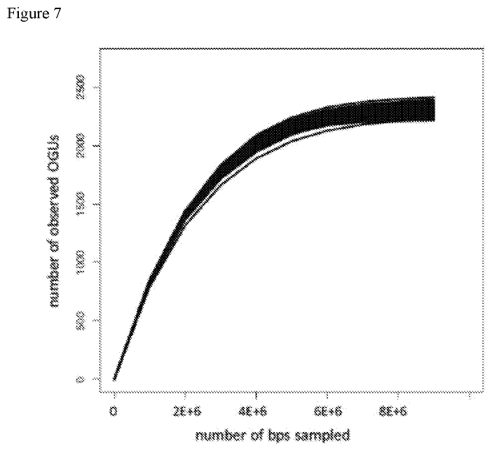

To better understand the role of the follicular microbiota in acne pathogenesis at the molecular level, the differences in metagenomic composition were investigated of the microbiome between acne patients and healthy individuals. Among the sequencing reads mapped to P. acnes, 2,707 operational gene units (OGUs) were identified in the samples, among which 1,943 OGUs (72%) were present in every sample. Rarefaction analysis indicated that the sequencing depth was sufficient to quantitatively compare the P. acnes functional profiles between acne patients and healthy individuals (FIG. 7). Since the microbiome profiles were similar between younger and older healthy individuals at both the species and P. acnes strain level, the healthy individuals from both age groups were combined in the following metagenomic analyses.

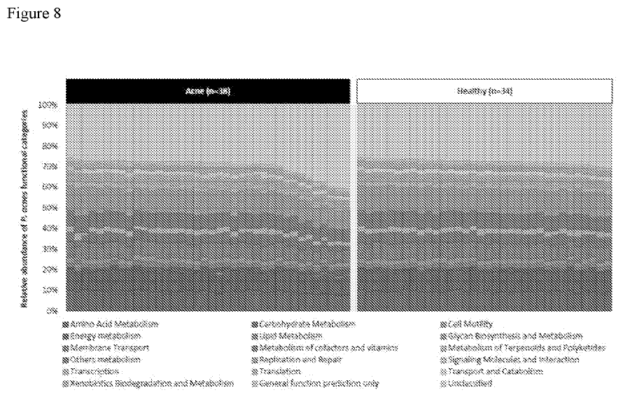

To determine whether specific metagenomic elements are associated with acne or healthy skin, the relative abundances of P. acnes OGUs were compared between acne patients and healthy individuals (FIG. 8). As the skin microbiome varies greatly among individuals, to reduce the potential bias due to specific individuals recruited in the study, 100 subsets of the data were randomly selected, each containing two-thirds of the individuals from each clinical group (26 acne patients and 22 healthy individuals), and determined the metagenomic elements that were differentially abundant between the two groups in each random set. To capture the differences at the species level between the two cohorts, in addition to P. acnes OGUs, the species found in the samples as additional metagenomic elements was considered and included them in the analysis. To determine the most robust set of metagenomic elements that are associated with either acne or healthy skin, 63 metagenomic elements whose relative abundances were significantly different (P<0.05) between the two groups in at least 50 of the 100 random subsets were identified. These 63 elements consisted of 62 P. acnes OGUs (Table 1) and P. granulosum species.

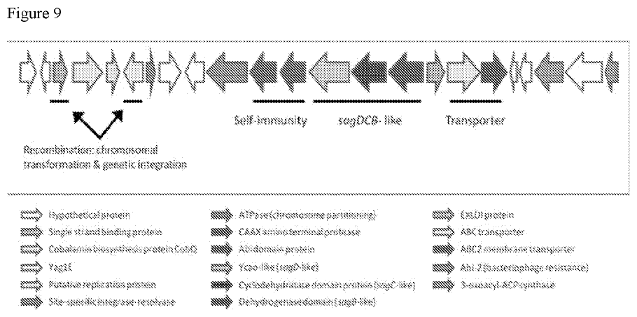

Among the 62 P. acnes OGUs, 25 were significantly more abundant in acne patients. Two of them are involved in thiopeptide bacteriocin precursor synthesis and transport (PAGK2104 and PAGK2105, as annotated in P. acnes HL096PA1 genome). Thiopeptide bacteriocins belong to a family of microcins, which are produced by Gram-positive bacteria and inhibit the growth of other Gram-positive species by blocking protein translation. Additionally, 19 of the 25 acne-associated OGUs were from locus 2 (PAGK0160-PAGK0178), a genomic island previously identified mainly in RT4 and RT5 strains and highly associated with acne. Locus 2 spans over 20 Kb and encodes 23 ORFs (FIG. 9). The differentially abundant locus 2 OGUs identified include a number of genetic elements involved in recombination, such as a single-strand binding like protein (Ssb), shown in other bacterial species to be involved in chromosomal transformation, and resolvase-like protein, previously implicated in genetic integration. Other OGUs include a cluster of Streptolysin S-associated genes (sag) involved in the biosynthesis and transport of a bacterial toxin. Identified and characterized in Streptococcus pyogenes, sag genes are involved in synthesis, post-translational modification, and transport of a ribosomally synthesized bacteriocin, which has been linked to antimicrobial activity as well as invasive infections. The presence of genes homologous to sagB, C and D in locus 2 suggests involvement of this locus in bacteriocin biosynthesis and maturation. Additionally self-immunity and transport genes are found immediately downstream. Other OGUs in this locus have putative roles in cell viability, virulence, and immunity including ATP-binding cassette (ABC) transporter and ABC-binding protein for translocation of lipids, nutrients and/or toxins, CAAX amino protease, which is thought to be involved in self-immunity, and partitioning machinery needed for cell replication and division. While the specific functions of genes in this locus in acne are unclear, the absence of locus 2 in healthy individuals and a high abundance in acne patients provides strong evidence for its association with acne.

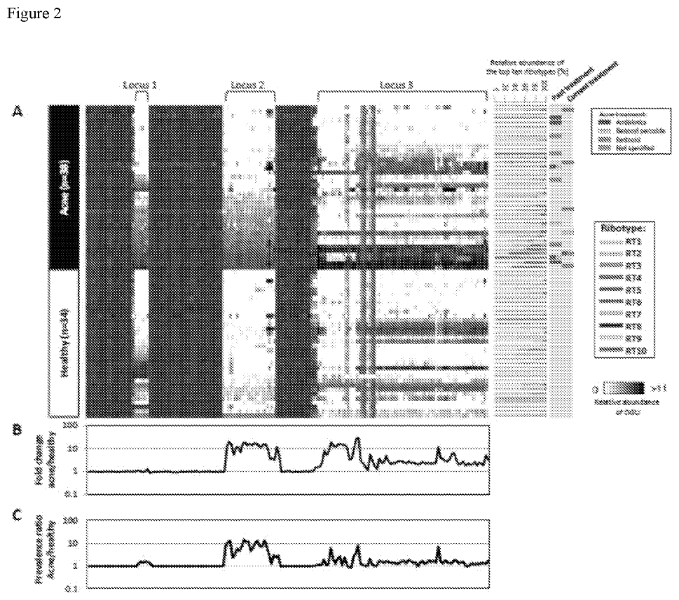

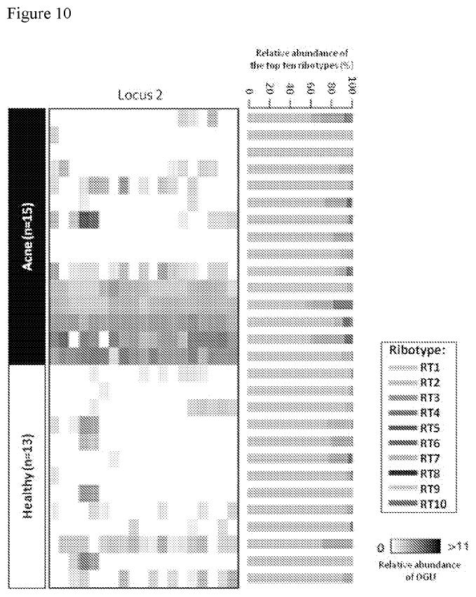

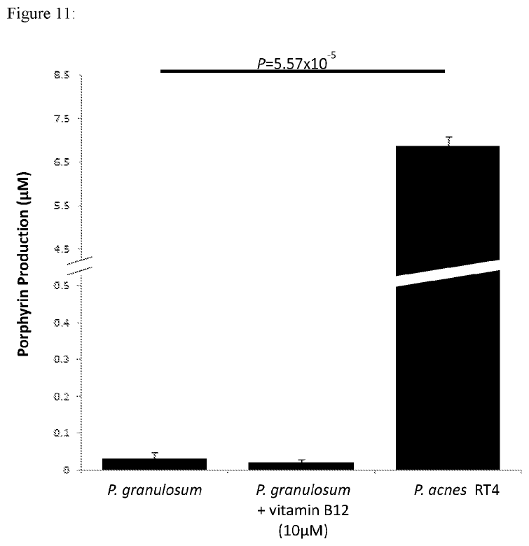

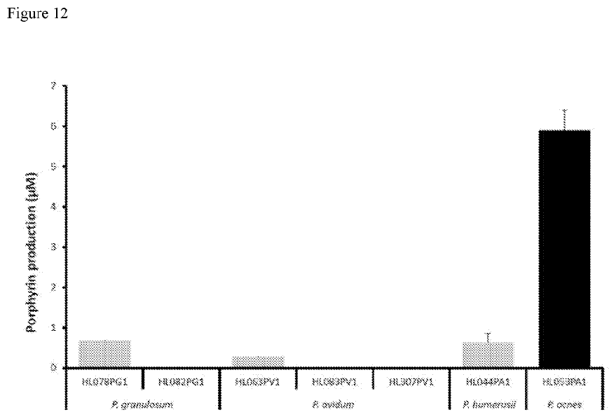

A higher average relative abundance and prevalence of all three loci was found in acne patients compared to healthy individuals (FIG. 2). Locus 2 was rarely found in the microbiome of the healthy individuals, and was significantly associated with acne (P=0.002) (FIG. 2). Since most RT4 and RT5 strains harbor locus 2 and are resistant to antibiotics, the treatment history of each individual was examined to determine whether the presence of locus 2 was due to acne treatment. While locus 2 was found in some treated acne patients (n=8), it was absent in other treated patients (n=8) and was present in many untreated patients (n=10). The presence of locus 2 was not significantly different between treated and untreated patients (Fisher's exact test, P=1), suggesting that locus 2 is a characteristic of the disease rather than treatment (FIG. 2, Part A). Additional analysis to further support the association of locus 2 with the disease is described in the supporting text (FIG. 10).

Of the 62 differentially abundant P. acnes OGUs, 37 OGUs were more abundant in healthy individuals. In contrast to the enrichment of virulence-related genes observed in the acne metagenome, genes involved in microbial metabolism and nutrient biosynthesis were significantly more abundant in the healthy metagenome (FIG. 8). Examples include glycosyl transferase (PAGK0136), D-alanine-D-alanine ligase (PAGK0821), and cobalamin-independent methionine synthase (PAGK1035), which are involved in polysaccharide, cell wall, and amino acid biosynthesis, respectively. An enrichment of these genes in the healthy metagenome is consistent with the metagenomic study by Mathieu et al., which suggested a functional role of the resident bacteria in healthy skin in the exploitation of compounds from the human skin including sugars, lipids, and iron. Other significantly more abundant OGUs in the healthy metagenome include glycerol uptake facilitator protein (PAGK2214), which aids in carbohydrate metabolism processes by transporting glycerol across the cytoplasmic membrane. Recently it was shown that fermentation of glycerol by P. acnes contributes to skin and host health via the production of short chain fatty acids (SCFA). SCFA including acetic acid, lactic acid, and propionic acid were shown to significantly decrease the colonization of skin-associated pathogens such as methicillin-resistant Staphylococcus aureus.

### Example 4: The Balance Between Acne- and Health-Associated Metagenomic Elements Shapes the Host Skin Microbiota in Acne and Health

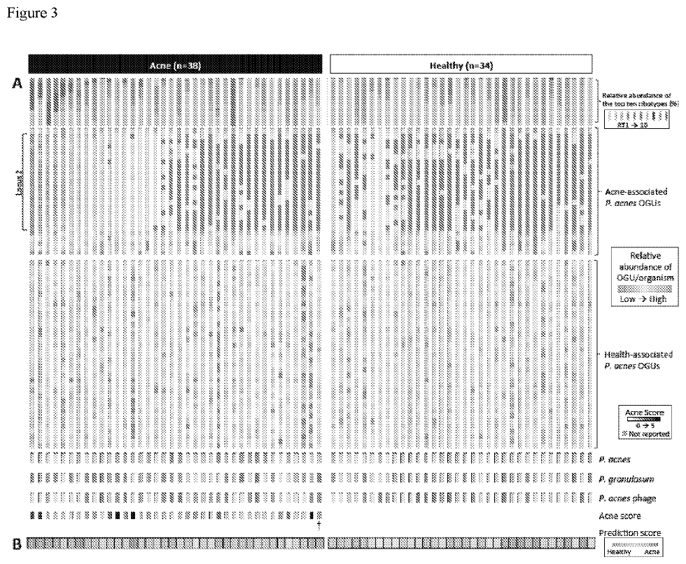

Observations of differentially abundant species, strains, and metagenomic elements between the skin microbiome of acne patients and healthy individuals led to the hypothesis that the balance between acne- and health-associated metagenomic elements determines the virulence and health properties of the microbiota in skin disease and health. The balance is defined as the ratio between the relative abundances of acne- and health-associated metagenomic elements. When the relative abundances of the 62 P. acnes OGUs and P. granulosum from all individuals were compared, the ratio in acne patients was significantly higher than in healthy individuals (P=2.9×10⁻⁵). FIG. 3 illustrates the relative abundances of these 63 metagenomic elements across all 72 individuals. The metagenomic profiles of healthy individuals were readily distinguishable from acne patients harboring locus 2. Acne patients with locus 2 had significantly higher relative abundances of acne-associated OGUs (in 24 of 25 OGUs P=0.00024-0.021, and in 1 of 25 OGUs P=0.083) (FIG. 3, Part A). However, these subjects only represented about half (47%) of the patients recruited in the study. When the metagenomic profiles of the other acne patients were examined without locus 2, it was found that they were distinct from healthy individuals with decreased relative abundances of health-associated P. acnes OGUs (in 23 of 37 OGUs P=0.0024-0.046, and in 14 of 37 OGUs P=0.058-0.30). Distinct microbiome profiles between acne patients and healthy individuals that were not readily apparent at the taxonomic level, with differences in the relative abundances of the metagenomic elements that were associated with either acne or healthy skin (FIG. 3).

In addition to P. acnes OGUs, it was found that the relative abundances of P. acnes and P. granulosum were also important factors influencing the clinical status of the skin. As an example, an acne patient in the cohort (labeled with † on FIG. 3) displayed a profile of the 62 P. acnes OGUs that was more similar to the healthy individuals than all other acne patients (P=5.29×10⁻⁵). However, the observed low relative abundances of both P. acnes (78% compared to the average of 91%) and P. granulosum (0.070% compared to the average of 1.6%) may contribute to the acne status of this patient. This suggests that a healthy follicular microbiota not only requires health-associated P. acnes strains and genetic elements, but also requires significant dominance of resident propionibacteria. Taken together, the findings suggest that the balance between skin metagenomic elements determines the virulence and health properties of the skin microbiota and is important in skin health and disease.

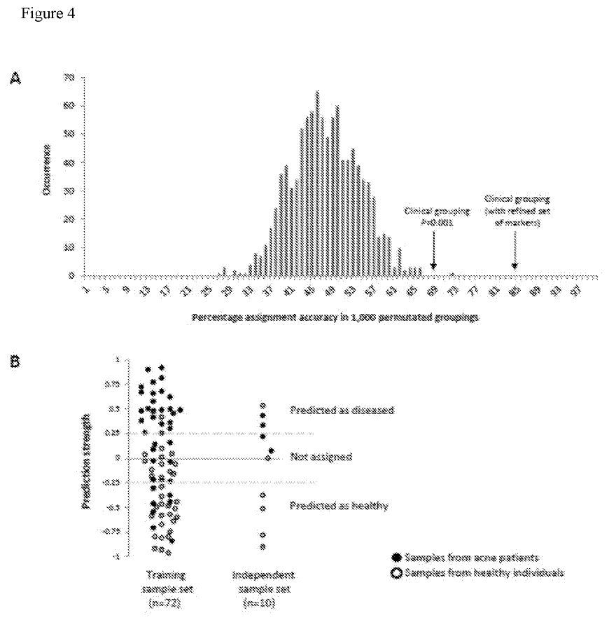

It was investigated whether the relative abundance profiles of the metagenomic elements are sufficiently robust to correctly classify the clinical status of skin samples. A supervised class performed prediction analysis based on a modified weighted gene voting algorithm. One thousand permutations were generated. In each permutation, samples were randomly assigned to either acne or healthy status, with 38 samples in the acne group and 34 samples in the healthy group. Differentially abundant metagenomic elements were identified between the two groups among all but one sample, and predicted the clinical state of the withheld sample based on the abundance profiles of the metagenomic element, known as the leave-one-out cross-validation (LOOCV). With the use of the clinically defined acne-healthy grouping, the algorithm predicted the clinical states of 49 out of 72 samples (68%) with a prediction strength threshold of 0.25. Thirty-four of the 49 samples were assigned correctly (69% accuracy). The number of correctly assigned samples based on the clinical grouping was higher than most of the permutated groupings (P=0.064). The clinical grouping also had a significantly higher prediction accuracy (69%) than all but one of the permutated groupings (P=0.001) (FIG. 4, Part A).

The classifier was improved by using the 63 metagenomic elements, which are the most robust set based on the 100 random samplings as described earlier. Since the 19 OGUs from locus 2 had similar abundance profiles across all samples, to avoid overweighing locus 2 in the classification, 19 OGUs were combined and used their average relative abundance in the classifier. The refined classifier therefore consisted of the abundance profiles of 45 metagenomic elements: P. acnes OGUs/loci, and P. granulosum. Based on this refined set, the clinical states of 31 of the 38 acne patients (82%, prediction strength threshold of 0.25) were assigned. Twenty-four acne patients were correctly assigned (accuracy of 77%). Among the healthy individuals, 21 of the 34 subjects (62%) were assigned with 20 correctly assigned (accuracy of 95%). Overall, the classifier was able to assign 72% of the subjects with an accuracy of 85% (FIG. 3 Part B; FIG. 4, Part B).

Furthermore, to validate that these metagenomic elements can be used as markers for clinical classification, an “independent sample set” was collected from ten additional subjects, including 4 acne patients and 6 healthy individuals, one of which was over 50 years old. The refined 45 metagenomic elements were used to predict the clinical state of each independent sample (FIG. 4, Part B). The classifier was able to assign the clinical states of 7 samples (70%) with an accuracy of 86%, highly consistent with the classification results from the training sample set of 72 samples. Based on this, it was concluded that the metagenomic elements identified in the study can be used to classify the clinical status of the skin with high accuracy.

A performed supervised class prediction analysis. Differentially abundant metagenomic elements, including P. acnes OGUs and bacterial species, were determined between two groups of samples (healthy and acne). A method similar to that described by Golub et al. Science 286, 531-537 (1999) and Bleharski et al. Science 301, 1527-1530, hereby incorporated by reference the their entirety, was employed, using the formula:

$$PS = \frac{{\sum }_{g = 1}^{n} {t}_{g} \times ( {X}_{g} - ( \frac{{μ}_{1 g} + {μ}_{2 g}}{2} ) )}{{\sum }_{g = 1}^{n} \vert {t}_{g} \times ( {X}_{g} - ( \begin{matrix}{μ}_{1 g} + {μ}_{2 g} \\ 2\end{matrix} ) ) \vert}$$

where PS is the prediction strength, a measure of the relative margin of victory of the vote, X_{g} is the relative abundance of the metagenomic element (g) in the tested sample, μ_{1g} and μ_{2g} are the means of the relative abundances of metagenomic element (g) in the two groups, and t_{g} is the t-test score of the metagenomic element (g) when its relative abundance is compared between the two groups using the Student's t-test. The numerator indicates the difference between the winning and losing classes, and the denominator indicates the totals for the winning and losing classes.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments are described herein. Such equivalents are intended to be encompassed by the following claims.

1. A method of treating or preventing a skin disease or skin aging in a subject, comprising administering a composition comprising Propionibacterium granulosum (P. granulosum), Propionibacterium avidum (P. avidum), or Propionibacterium humerusii (P. humerusii), or a combination thereof, to the subject.

2. The method of claim 1, wherein the skin disease is an inflammatory skin disease.

3. The method of claim 1, wherein the skin disease comprises acne, aging, inflammation, rosacea, or Porphyria Cutanea Tarda (PCT), or a combination thereof.

4-5. (canceled)

6. The method of claim 1, wherein the administration reduces the amount of porphyrins on the skin of a subject.

7-11. (canceled)

12. The method of claim 1, wherein the composition further comprises one or more strains of P. acnes.

13. The method of claim 12, wherein the P. acnes strain comprises a RT1, RT2, RT3, or RT6 strain of P. acnes, or a combination thereof.

14. The method of claim 1, comprising administering at least one phage against a strain of P. acnes to the subject.

15. The method of claim 14, wherein the phage targets a strain of P. acnes comprising RT4, RT5, RT7, RT8, RT9 or RT10, or a combination thereof.

16. The method of claim 1, comprising administering an antibiotic to the subject.

17-34. (canceled)

35. The method of claim 1, wherein: (a) the composition comprises P. granulosum and wherein the P. granulosum comprises strain HL078PG1 and/or HL082PG1, (b) the composition comprises P. avidum and the wherein the P. avidum comprises strain HL063PV1, HL083PV1 or HL307PV1, or a combination thereof, (c) the composition comprises P. humerusii and wherein the P. humerusii comprises strain HL044PA1, (d) a combination of (a) and (b), (e) a combination of (a) and (c), (f) a combination of (b) and (c), or (g) a combination of (a), (b), and (c).

36-40. (canceled)

41. A pharmaceutical or cosmetic composition comprising at least one strain of P. granulosum, at least one strain of P. avidum, or at least one strain of P. humerusii, or a combination thereof.

42. The composition of claim 41, wherein the composition further comprises at least one strain of P. acnes.

43. The composition of claim 42, wherein the at least one strain of P. acnes comprises RT1, RT2, RT3, or RT6, or a combination thereof.

44. The composition of claim 43, wherein the P. acnes is enriched for one or more of the genes associated with healthy skin in Table 1.

45. The composition of claim 41, wherein the composition comprises a phage against RT4, RT5, RT7, RT8, RT9 or a RT10 strain of P. acnes, or a combination thereof.

46. The composition of claim 41, comprising the at least one strain of P. granulosum, and wherein the P. granulosum strain comprises HL078PG1 and/or HL082PG1.

47-56. (canceled)

57. The composition of claim 41, comprising the at least one strain of P. avidum, and wherein the P. avidum comprises strain HL063PV1, HL083PV1, HL307PV1, or a combination thereof.

58-60. (canceled)

61. The composition of claim 41, comprising the at least one strain of P. humerusii, and wherein the P. humerusii comprises strain HL044PA1.

62-76. (canceled)

77. The composition of claim 41, comprising one or more strains of P. acnes.

78. (canceled)

79. The composition of claim 41, comprising at least one phage against a strain of P. acnes.

80-98. (canceled)
